(19) United States
(12) Patent Application Publication
(10) Pub. No.: US 2026/0090997 A1
(43) Pub. Date: Apr. 2, 2026

(54) Lipid Nanoparticles Comprising Nucleic Acids Encoding Therapeutic Genes and Uses in Medical Methods

(71) Applicants: Emory University, Atlanta, GA (US); Children's Healthcare of Atlanta, Inc., Atlanta, GA (US); Georgia Tech Research Corporation, Atlanta, GA (US)

(72) Inventors: Eric J. Sorscher, Atlanta, GA (US); Jeong S. Hong, Atlanta, GA (US); Annette Ehrhardt, Atlanta, GA (US); Regina Rab, Atlanta, GA (US); Bhagelu Achyut, Atlanta, GA (US); James E. Dahlman, Atlanta, GA (US); Melissa P. Lokugamage, Atlanta, GA (US); Sebastian G. Huayamares, Atlanta, GA (US); Philip John Santangelo, Atlanta, GA (US)

(21) Appl. No.: 19/113,702

(22) PCT Filed: Sep. 20, 2023
(86) PCT No.: PCT/US2023/033245

§ 371 Date: Mar. 20, 2025

Related U.S. Application Data
(60) Provisional application No. 63/408,343, filed on Sep. 20, 2022.
(60) Provisional application No. 63/468,352, filed on May 23, 2023.

(51) Int. Cl. A61K 9/51; A61K 38/00; C07K 16/28; C12N 9/10; C12N 9/24; C12N 15/88

(52) U.S. Cl. CPC A61K 9/5123; C07K 16/2818; C12N 9/1077; C12N 9/2497; C12N 15/88; A61K 38/00; C07K 2317/569; C12Y 204/02001; C12Y 302/02001

(57) ABSTRACT

This disclosure relates to lipid nanoparticles comprising nucleic acids encoding therapeutic proteins and uses in treating diseases such as cancer. In certain embodiments, this disclosure relates to methods of treating cancer or initiating, enhancing, or prolonging an anti-tumor response in a subject in need thereof comprising administering to the subject an effective amount of lipid nanoparticles as reported herein comprising a vector or nucleic acid encoding peptide based anticancer agent.

### CROSS-REFERENCE TO RELATED APPLICATIONS

This application claims the benefit of U.S. Provisional Application No. 63/468,352 filed May 23, 2023, and U.S. Provisional Application No. 63/408,343 filed Sep. 20, 2022. The entirety of each of these applications is hereby incorporated by reference for all purposes.

### STATEMENT REGARDING FEDERALLY SPONSORED RESEARCH OR DEVELOPMENT

This invention was made with government support under DE026941 awarded by the National Institutes of Health. The government has certain rights in the invention.

### BACKGROUND

End-stage cancer, e.g., head and neck squamous cell carcinoma (HNSCC), is typically associated with severe morbidity, chronic pain, and uncontrolled bleeding, among other symptoms. Unfortunately, the use of chemotherapy or external beam radiation therapy to treat HNSCC is not universally effective. Thus, there is a need to identify improved therapies.

Fludarabine is a clinically approved chemotherapeutic that can be converted in vivo into fludarabine triphosphate. In cells with E. coli purine nucleoside phosphorylase (PNP), fludarabine can be converted into F-ATP. When F-ATP is incorporated into RNA it disrupts RNA and protein synthesis. Anti-tumor strategies utilizing E. coli purine nucleoside phosphorylase (PNP) to activate purine nucleoside analogs have been reported. See, e.g., Sorscher et al. which report that systemically administered fludarabine phosphate (F-araAMP) slows growth of human tumor xenografts that express E. coli PNP. Cancer Chemother Pharmacol, 2012, 70 (2): 321-329. See also WO2021183640.

Dahlman et al. report barcoded nanoparticles for high throughput in vivo discovery of targeted therapeutics. PNAS, 2017, 114 (8) 2060-2065. See also e.g., PCT WO2021021636.

References cited herein are not an admission of prior art.

### SUMMARY

This disclosure relates to lipid nanoparticles comprising nucleic acids encoding therapeutic proteins and uses in treating diseases such as cancer. In certain embodiments, this disclosure relates to methods of treating cancer or initiating, enhancing, or prolonging an anti-tumor response in a subject in need thereof comprising administering to the subject an effective amount of lipid nanoparticles as reported herein comprising a vector or nucleic acid encoding peptide based anticancer agent.

In certain embodiments, the peptide based anticancer agent is an antibody, antibody fragment, single chain antibody that specifically binds a cancer antigen. In certain embodiments, the peptide, antibody, antibody fragment, or single chain antibody is fused to a glycosylphosphatidylinositol (GPI) membrane anchor sequence.

In certain embodiments, this disclosure relates to methods of treating cancer comprising administering effective amounts of nanoparticles disclosed herein comprising a single chain antibody that specifically binds a cancer antigen. In certain embodiments, the peptide based anticancer agent such as a single chain antibody is administered systemically.

In certain embodiments, contemplated nanoparticles comprise a nucleic acid, such as mRNA or DNA, that encodes a therapeutic peptide, an ionizable lipid, a phospholipid, a sterol, and a polyethylene glycol phospholipid. In certain embodiments, the ionizable lipid is 7C1 of about between 35-52% of the lipid nanoparticle by molar ratio, wherein the phospholipid is distearoylphosphatidylcholine (DSPC) of about between 15-25% of the lipid nanoparticle by molar ratio, wherein the sterol is cholesterol of about between 15-45% of the lipid nanoparticle by molar ratio, and wherein the polyethylene glycol phospholipid is poly(ethylene glycol)-distearoylphosphatidylethanolamine of about between 1-15% of the lipid nanoparticle by molar ratio and wherein the polyethylene glycol chains have an average molecule weight average molecular weight of about between 1400 and 2800 Da.

In certain embodiments, contemplated nanoparticles comprise a nucleic acid, such as mRNA or DNA, that encodes a therapeutic peptide, an ionizable lipid, a phospholipid, a sterol, and a polyethylene glycol phospholipid. In certain embodiments, the ionizable lipid is 7C1 of about between 48-52% of the lipid nanoparticle by molar ratio, wherein the phospholipid is distearoylphosphatidylcholine (DSPC) of about between 14-16% of the lipid nanoparticle by molar ratio, wherein the sterol is cholesterol of about between 27-30% of the lipid nanoparticle by molar ratio, and wherein the polyethylene glycol phospholipid is poly(ethylene glycol)-distearoylphosphatidylethanolamine of about between 5-7% of the lipid nanoparticle by molar ratio and wherein the polyethylene glycol chains have an average molecule weight average molecular weight of about between 1900 and 2100 Da.

In certain embodiments, this disclosure relates to methods of treating cancer comprising administering effective amounts of nanoparticles disclosed herein that contain a nucleic acid, e.g., mRNA or DNA, that encodes a peptide based anticancer agent which is a purine nucleoside cleaving enzyme and administering a prodrug cleaved by said purine nucleoside cleaving enzyme to a subject in need thereof. In certain embodiments, the purine nucleoside cleaving enzyme is a non-mammalian purine nucleoside phosphorylase (PNP) or nucleoside hydrolase (NH). In certain embodiments, the nanoparticles are administered intratumorally or local to the area or tissue surrounding a tumor.

In certain embodiments, contemplated nanoparticles comprise a nucleic acid, such as mRNA or DNA, that encodes a therapeutic peptide, an ionizable lipid, a phospholipid, a sterol, and a polyethylene glycol phospholipid. In certain embodiments, the ionizable lipid is (2S,2′S)-1,1′-((2-(((S)-2-hydroxydodecyl)(2-(4-(2-(((S)-2-hydroxydodecyl)((S)-2-hydroxydodecyl)amino)ethyl)piperazin-1-yl)ethyl)amino)ethyl)azanediyl)bis(dodecan-2-ol) (KB-S) or 2R,2′R)-1,1′-((2-(((R)-2-hydroxydodecyl)(2-(4-(2-(((R)-2-hydroxydodecyl)((R)-2-hydroxydodecyl)amino)ethyl)piperazin-1-yl)ethyl)amino)ethyl)azanediyl)bis(dodecan-2-ol) (KB-R) of between 32-34% of the lipid nanoparticle by molar ratio; the phospholipid is 1,2-dioleoyl-sn-glycero-3-phosphoethanolamine (DOPE) of between 45-47% of the lipid nanoparticle by molar ratio; wherein the sterol is cholesterol of between 17-19% of the lipid nanoparticle by molar ratio; and the polyethylene glycol phospholipid is poly(ethylene glycol)-distearoyl phosphatidylethanolamine derivative of between 2-4% by molecular ratio of the lipid nanoparticle wherein the polyethylene glycol phospholipid has polyethylene glycol chains with an average molecule weight of about between 1900 and 2100 Da.

In certain embodiments, contemplated nanoparticles comprise a nucleic acid, such as mRNA or DNA, that encodes a therapeutic peptide, an ionizable lipid, a phospholipid, a sterol, and a polyethylene glycol phospholipid. In certain embodiments, the ionizable lipid is (2S,2′S)-1,1′-((2-(((S)-2-hydroxydodecyl)(2-(4-(2-(((S)-2-hydroxydodecyl)((S)-2-hydroxydodecyl)amino)ethyl)piperazin-1-yl)ethyl)amino)ethyl)azanediyl)bis(dodecan-2-ol) (KB-S) or 2R,2′R)-1,1′-((2-(((R)-2-hydroxydodecyl)(2-(4-(2-(((R)-2-hydroxydodecyl)((R)-2-hydroxydodecyl)amino)ethyl)piperazin-1-yl)ethyl)amino)ethyl)azanediyl)bis(dodecan-2-ol) (KB-R) of between 32-34% of the lipid nanoparticle by molar ratio; the phospholipid is 1,2-dioleoyl-sn-glycero-3-phosphoethanolamine (DOPE) of between 45-47% of the lipid nanoparticle by molar ratio; wherein the sterol is cholesterol of between 17-19% of the lipid nanoparticle by molar ratio; and the polyethylene glycol phospholipid is poly(ethylene glycol)-distearoyl phosphatidylethanolamine derivative of between 2-4% by molecular ratio of the lipid nanoparticle wherein the polyethylene glycol phospholipid has polyethylene glycol chains with an average molecule weight of about between 1400 and 2800 Da.

In certain embodiments, the peptide based anticancer agent is a purine nucleoside cleaving enzyme and administering a prodrug cleaved by said purine nucleoside cleaving enzyme to the subject. In certain embodiments, the purine nucleoside cleaving enzyme is a non-mammalian purine nucleoside phosphorylase (PNP) or nucleoside hydrolase (NH). In certain embodiments, the prodrug is 9-(β-D-arabinofuranosyl)-2-fluoroadenine (F-araA), 2-F-2′-deoxyadenosine (F-dAdo), fludarabine phosphate (F-araAMP, 2-fluoro-9-(5-O-phosphono-β-D-arabinofuranosyl)-9H-purin-6-amine), derivative, or salt thereof.

In certain embodiments, any of the methods disclosed herein are done in combination with administering another anticancer agent to the subject, radiation therapy, surgical interventions, or combinations thereof.

In certain embodiments, nanoparticles disclosed herein are used in the production of a medicament for treating cancer.

### BRIEF DESCRIPTION OF THE SEVERAL VIEWS OF THE DRAWINGS

FIGS. 1A-1F illustrate a high-throughput DNA barcoding method used to study the delivery of LNPs to tumor cells in vivo.

FIG. 1A illustrates the chemical structures of components used to create a diverse library of LNPs formulated using 7C1, cholesterol, DSPC, and PEG-lipid variants at various mole ratios.

FIG. 1B illustrates that each LNP was formulated to carry a distinct DNA barcode.

FIG. 1C illustrates a method where small, monodisperse LNPs are pooled together and administered into a mouse to find an LNP that is preferentially delivered to human cancer cells. Human head and neck cancer cells (FADU or Cal-27) were subcutaneously injected into Nu/J mice to create xenograft hindleg tumors. Pooled LNP libraries were intravenously administered to mice. Twenty-four hours later, human cancer cells and liver hepatocytes were isolated for sequencing.

FIG. 1D shows data on normalized delivery of LNPs in tumor cells and hepatocytes. The open dot represents unencapsulated DNA barcode, which did not deliver well to either cell type and acts as a control showing that certain LNPs increase delivery of barcodes indicating a correlation between delivery for LNPs in mouse hepatocytes and human tumor cells.

FIG. 1E shows data in FADU tumor cells.

FIG. 1F shows data for Cal-27 tumor cells.

FIG. 2A illustrates components used in follow-on high-throughput LNP screens designed to identify an LNP that preferentially delivered to human tumor cells in vivo. Enrichment in the top 5% of LNPs, subdivided by the PEG type and mole ratio of 7C1 suggests that C18PEG2000 with a 50% mole ratio were enriched in human tumor cells.

FIG. 2B shows data on normalized delivery of LNPs in tumors and hepatocytes. The negative control (circle) was lower than normalized delivery for all LNPs.

FIG. 2C shows data on the delivery of LNPs in mouse hepatocytes and human FADU tumor cells.

FIG. 2D shows data in Cal-27 tumor cells.

FIG. 3A illustrates the TL1 (tumor lipid nanoparticle 1) formulated with a GFP mRNA and an anchored single heavy chain antibody with a GPI membrane-anchor VHH (a VHH) mRNA.

FIG. 3B shows data where FADU cancer cells were transfected with TL1 (tumor lipid nanoparticle 1) containing GFP mRNA in vitro at 0.5, 1, and 2 μg/80M cells.

FIG. 3C shows data wherein aVHH expression was quantified via flow cytometry. Transfection with TL1 (tumor lipid nanoparticle 1) containing mRNA encoding aVHH in FADU cells was also validated via immunohistochemical staining and microscopy. TL1 functionally delivers various mRNA payloads to tumor cell types.

FIG. 4A illustrates a method where TL1 is formulated with an anchored NanoLuc mRNA and administered intravenously to Nu/J mice with FADU xenograft tumors at 1.5 mg of RNA/kg (mouse).

FIG. 4B shows data where after twenty-four hours, luciferase expression was quantified via whole-organ imaging in FADU bi-lateral tumors.

FIG. 4C illustrates a method using TL1 with an anchored NanoLuc mRNA and administered intravenously to NSG mice with PDX tumors at 1.5 mg of RNA/kg of mouse.

FIG. 4D shows data where luciferase expression was quantified via whole-organ imaging in PDX bi-lateral tumors.

FIG. 5A illustrates different LNPs formulated to carry NanoLuc reporter mRNA to evaluate effective intratumoral injections into FADU xenografts. To identify the most suitable ionizable lipid for intratumoral delivery, an in vivo imaging system was used to quantify the bioluminescence resulting from the functional delivery of NanoLuc mRNA using LNPs containing four different ionizable lipids: MC3, cKK-E12, SM-102, and C12-200.

FIG. 5B shows data indicating C12-200 was an optimal ionizable lipid from a high-throughput screen.

FIG. 5C illustrates LNPs formulated to carry a distinct DNA barcode and a VHH reporter mRNA.

FIG. 5D illustrates a method where small, monodisperse LNPs are pooled together and administered into a mouse. Human head and neck cancer cells (FADU) were subcutaneously injected into Nu/J mice to induce xenograft hindleg tumors. Pooled LNP libraries were intratumorally administered to mice. Sixteen hours later, once the transfected cells expressed a VHH, FADU tumors were isolated for sequencing.

FIG. 5E illustrates C12-200 stereo-pure derivates (KB-S, KB-R, with various alkyl chains) which were used to formulate various LNPs.

FIG. 5F illustrates other LNP components DOTAP and DC-Cholesterol hydrochloride that were evaluated in addition to C18PEG2K-lipid, cholesterol (FIG. 1A) and DOPE (FIG. 2A).

FIG. 5G shows the hydrodynamic diameter of administered LNPs.

FIG. 5H shows normalized delivery of LNPs in tumor cells.

FIG. 6A shows data on the enrichment in the top 12% of LNPs screened, subdivided by the type of cholesterol, helper lipid, and stereo-pure ionizable lipid (KB lipid).

FIG. 6B shows percentages of components of the most desirable LNPs (LNP 28 and LNP 49) that were identified from the screen based on the enrichment of the stereo-pure ionizable lipids and other components.

FIG. 6C shows data indicating both LNP 28 and LNP 49 functionally deliver NanoLuc mRNA when injected intratumorally to the tumors as demonstrated by the quantification of bioluminescence.

FIG. 6D shows data indicating LNP 28 and LNP 49 functionally delivered a second reporter mRNA tested (aVHH) to CD47+ human head & neck cancer cells (FADU) and various infiltrating immune cells in the tumors, as quantified via flow cytometry. ECs: Endothelial cells.

FIG. 7A illustrates patient-derived xenografts (PDX) (induced in NSG mice) which were then injected intratumorally with LNP 28 and LNP 49 to test the delivery of two mRNAs: NanoLuc and a VHH.

FIG. 7B shows quantified bioluminescence indicating that LNP 49 achieved optimal functional delivery of NanoLuc in PDX tumors via intratumoral injection.

FIG. 7C shows data indicating LNP 49 functionally delivered aVHH to about 22% of PDX tumor cells, as well as multiple different immune cell types. ECs: Endothelial cells. LNP 49 functionally delivers two different reporter mRNA molecules to PDX tumor cell types in vivo.

FIG. 8A illustrates a LNP (IT LNP) containing mRNA encoding E. coli Purine Nucleoside Phosphorylase (PNP) for transfection and expression in human PDX tumors. Contemplated is a cytoreductive mechanism using IT LNP-PNP in combination with Fludarabine. IT LNP is used to deliver E. coli Purine Nucleoside Phosphorylase (PNP)-encoding mRNA to human HNSCC cancer cells. An HPLC-based PNP enzymatic assay was used to measure PNP expression in transfected PDX tumors.

FIG. 8B shows data on PNP activity in PDX tumors harvested at different timepoints to determine the pharmacokinetics of the PNP mRNA. Dose response of the IT LNP-PNP when concentrated and administered at 40 μg per PDX tumor is shown.

FIG. 8C shows data indicating IT LNPs carrying PNP mRNA, combined with Fludarabine, can confer antitumor activity against HNSCC patient-derived xenografts. PDX tumors were inoculated in NSG mice. On day 14, IT LNP-PNP (or PBS for control groups) was injected at 20 μg/tumor in the morning and in the afternoon (40 μg total). The next day, Fludarabine (arrow) or DMSO was injected in the morning and afternoon, and tumor volumes were measured twice a week. Tumor growth was strongly inhibited for the group treated with IT LNP-PNP & Fludarabine, compared to the control groups. Representative mice from groups 1 and 4 showed size reduction of the treated PDX tumor on day 16. When two doses of IT LNP-PNP & Fludarabine were administered, 5 of the 6 mice showed complete tumor regression.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims. “Embodiments” refer to an example, and it is contemplated that the embodiments are not necessarily limited to the example.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent. Also, the term “or” is used in its inclusive sense (and not in its exclusive sense) so that when used, for example, to connect a list of elements, the term “or” means one, some, or all of the elements in the list.

As used herein, the term “7C1” refers to a polyethyleneimine polymer (PEI600)amino substituted with pentadecan-2-ol groups illustrated by a chemical structure:

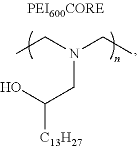

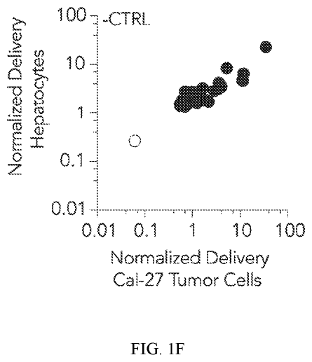

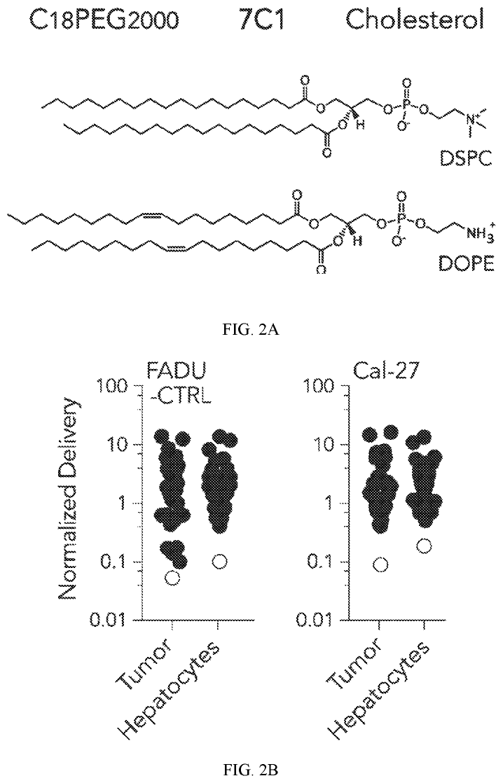

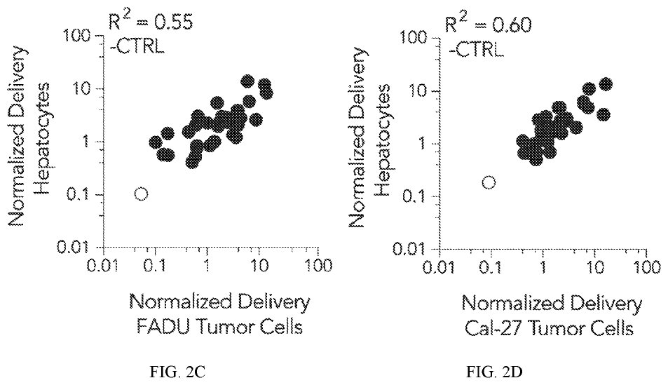

as reported in FIG. 2, Panel A of Dahlman et al. entitled “Barcoded nanoparticles for high throughput in vivo discovery of targeted therapeutics,” PNAS, 2017, 114 (8) 2060-2065 incorporated by reference in its entirety. The polymer 7C1 is produced by contacting a 2-tridecyloxirane with PEI600 and heating to 90° C. in 100% ethanol for 48-72 hours as reported in Dahlman et al., entitled “In vivo endothelial siRNA delivery using polymeric nanoparticles with low molecular weight,” Nat Nanotechnol, 9 (2014) 648-655, hereby incorporated by reference in its entirety. 7C1 has an average molecular weight of about 3600 Da.

As used herein, the term “about” is synonymous with the term “approximately.” Illustratively, the use of the term “about” indicates that a value includes values slightly outside the cited values. Variation may be due to conditions such as experimental error, manufacturing tolerances, variations in equilibrium conditions, and the like. In some embodiments, the term “about” includes the cited value plus or minus 10%. In all cases, where the term “about” has been used to describe a value, it should be appreciated that this disclosure also supports the exact value.

The terms “comprising,” “including,” “having,” and the like are synonymous and are used inclusively, in an open-ended fashion, and do not exclude additional elements, features, acts, operations, and so forth.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein.

“Subject” refers to any animal, preferably a human patient, livestock, rodent, monkey, or domestic pet.

As used herein, the terms “treat” and “treating” are not limited to the case where the subject (e.g., patient) is cured and the disease is eradicated. Rather, embodiments of the present disclosure also contemplate treatment that merely reduces symptoms, and/or delays disease progression.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other such that they are contained/circulating in the patient at the same time, e.g., considering half-lives.

The term “nucleic acid” refers to a polymer of nucleotides, or a polynucleotide, e.g., RNA, DNA, or a combination thereof. The term is used to designate a single molecule, or a collection of molecules. Nucleic acids may be single stranded or double stranded and may include coding regions and regions of various control elements.

The term “encoding” refers to the inherent property of specific sequences of nucleotides in a polynucleotide, such as a gene, a cDNA, or an mRNA, to serve as templates or provide additional features important to synthesis of other polymers and macromolecules in biological processes having either a defined sequence of nucleotides (e.g., IRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence (with T replaced by U) and is usually provided in sequence listings, and the non-coding strand, used as the template for transcription of a gene or cDNA, can be referred to as encoding the protein or other product of that gene or cDNA.

As used herein, an “RNA” refers to a polymer of ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the nonlimiting group consisting of small interfering RNA (siRNA), microRNA (miRNA), Dicer-substrate RNA (dsRNA), small hairpin RNA (shRNA), mRNA, and mixtures thereof.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

A “heterologous” nucleic acid sequence or peptide sequence refers to a nucleic acid sequence or a peptide sequence that does not naturally occur, e.g., because the whole sequence contains a segment from other plants, bacteria, viruses, other organisms, or joinder of two sequences that occur in the same organism but are joined together in a manner that does not naturally occur in the same organism or any natural state.

The term “recombinant” when made in reference to a nucleic acid molecule refers to a nucleic acid molecule which is comprised of segments of nucleic acid joined together by means of molecular biological techniques provided that the entire nucleic acid sequence does not occur in nature, i.e., there is at least one mutation in the overall sequence such that the entire sequence is not naturally occurring even though separately segments may occur in nature. The segments may be joined in an altered arrangement such that the entire nucleic acid sequence from start to finish does not naturally occur. The term “recombinant” when made in reference to a protein or a peptide refers to a protein molecule that is expressed using a recombinant nucleic acid molecule.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular or cell-free expression systems. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In certain embodiments, this disclosure contemplates a vector encoding a peptide disclosed herein in operable combination with a heterologous promoter.

In certain contexts, an “antibody” refers to a protein-based molecule that is naturally produced by animals in response to the presence of a protein or other molecule or that is not recognized by the animal's immune system to be a “self” molecule, i.e., recognized by the animal to be a foreign molecule, i.e., an antigen to the antibody. The immune system of the animal will create an antibody to specifically bind the antigen (or any cell or organism attached to the antigen) and thereby targeting the antigen for degradation or elimination. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, the term “antibody” is intended to include natural antibodies, monoclonal antibody, or non-naturally produced synthetic antibodies, such as specific binding single chain antibodies, bispecific antibodies, or fragments thereof. These antibodies may have chemical modifications. The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same antigen. The term “monoclonal” is not limited to any particular method for making the antibody, nor is the term limited to antibodies produced in a particular species, e.g., mouse, rat, etc.

In humans, from a structural standpoint, an antibody is a combination of proteins: two heavy chain proteins and two light chain proteins. Alternatively, other animals produce antibodies from nucleic acids that encode a single protein. In humans, the heavy chains are longer than the light chains. The two heavy chains typically have the same amino acid sequence. Similarly, the two light chains typically have the same amino acid sequence. Each of the heavy and light chains contain a variable segment that contains amino acid sequences which participate in binding to the antigen. The variable segments of the heavy chain do not have the same amino acid sequences as the light chains. The variable segments are often referred to as the antigen binding domains. The antigen and the variable regions of the antibody may physically interact with each other at specific smaller segments of an antigen often referred to as the “epitope.” Epitopes usually consist of surface groupings of molecules, for example, amino acids or carbohydrates. The terms “variable region,” “antigen binding domain,” and “antigen binding region” refer to that portion of the antibody molecule which contains the amino acid residues that interact with an antigen and confer on the antibody its specificity and affinity for the antigen. Small binding regions within the antigen-binding domain that typically interact with the epitope are also commonly alternatively referred to as the “complementarity-determining regions, or CDRs.”

A “chimeric antibody” is a molecule in which different portions of the antibody are derived from different immunoglobulin molecules such that the entire molecule is not naturally occurring. Examples of chimeric antibodies include those having a variable region derived from a non-human antibody and a human immunoglobulin constant region. The term is also intended to include antibodies having a variable region derived from one human antibody grafted to an immunoglobulin constant region of a predetermined sequence or the constant region from another human for which there are allotypic differences residing in the constant regions of any naturally occurring antibody having the variable regions, e.g., CDRs 1, 2, and 3 of the light and heavy chain. Human heavy chain genes exhibit structural polymorphism (allotypes) that are inherited as a haplotype. The serologically defined allotypes differ within and between population groups. See Jefferis et al. mAb, 1 (2009), pp. 332-338.

“Single chain antibodies” refer to a single peptide containing naturally or non-naturally occurring sequences, including synthetically modified peptide sequences, derived from an antibody variable region that specifically binds an antigen of interest. Single chain antibodies are sometimes fragments or variants of naturally occurring mammalian antibodies. Such antibodies are sometimes referred to as single-domain antibodies (sdAbsor or VHHs), or camelid single-domain antibodies, e.g., when derived from an animal of Camelidae family, e.g., lamas, camels.

### Lipid Nanoparticles

In certain embodiments, lipid nanoparticles disclosed herein include ionizable lipids, PEG lipids, phospholipids, and sterols. In certain embodiments, lipid nanoparticles disclosed herein comprise a nucleic acid that encodes a therapeutic protein, an ionizable lipid, a phospholipid, a sterol, and a polyethylene glycol phospholipid.

In certain embodiments, the ionizable lipid is about between 35-65% of the lipid nanoparticle by molecular ratio, wherein the phospholipid of about between 0-30% of the lipid nanoparticle by molecular ratio, wherein the sterol of about between 15-65% of the lipid nanoparticle by molecular ratio, and wherein the polyethylene glycol phospholipid is about between 1-15% of the lipid nanoparticle.

In certain embodiments, the ionizable lipid is 7C1 of about between 35-50% of the lipid nanoparticle by molecular ratio, wherein the phospholipid is distearoylphosphatidylcholine (DSPC) of about between 15-25% of the lipid nanoparticle by molecular ratio, wherein the sterol is cholesterol of about between 15-45% of the lipid nanoparticle by molecular ratio, and wherein the polyethylene glycol phospholipid is poly(ethylene glycol)-distearoylphosphatidylethanolamine of about between 1-15% of the lipid nanoparticle and wherein the polyethylene glycol chains have an average molecular weight of about between 1400 and 2800 Da.

In certain embodiments, the ionizable lipid is about between 48-52% of the lipid nanoparticle by molecular ratio, wherein the phospholipid of about between 14-16% of the lipid nanoparticle by molecular ratio, wherein the sterol of about between 27-30% of the lipid nanoparticle by molecular ratio, and wherein the polyethylene glycol phospholipid is about between 5-7% of the lipid nanoparticle.

In certain embodiments, the ionizable lipid is 7C1 of about between 48-52% of the lipid nanoparticle by molecular ratio, wherein the phospholipid is distearoylphosphatidylcholine (DSPC) of about between 14-16% of the lipid nanoparticle by molecular ratio, wherein the sterol is cholesterol of about between 27-30% of the lipid nanoparticle by molecular ratio, and wherein the polyethylene glycol phospholipid is poly(ethylene glycol)-distearoylphosphatidylethanolamine of about between 5-7% of the lipid nanoparticle and wherein the polyethylene glycol chains have an average molecular weight of about between 1900 and 2100 Da.

In certain embodiments, the therapeutic protein is a single chain antibody.

In certain embodiments, the ionizable lipid is 7C1 or alternative.

In certain embodiments, the ionizable lipid is about between 40-60% of the lipid nanoparticle by molecular ratio, or about between 45-55% of the lipid nanoparticle by molecular ratio, or about between 48-52% of the lipid nanoparticle by molecular ratio.

In certain embodiments, the phospholipid is distearoylphosphatidylcholine (DSPC) or alternative.

In certain embodiments, the phospholipid is about between 10-20% of the lipid nanoparticle by molecular ratio, or about between 12-17% of the lipid nanoparticle by molecular ratio, or about between 14-16% of the lipid nanoparticle by molecular ratio.

In certain embodiments, the sterol is cholesterol or alternative.

In certain embodiments, the sterol is about between 25-32% of the lipid nanoparticle by molecular ratio, or about between 26-31% of the lipid nanoparticle by molecular ratio, or about between 27-30% of the lipid nanoparticle by molecular ratio.

In certain embodiments, the polyethylene glycol phospholipid is poly(ethylene glycol)-distearoylphosphatidylethanolamine (C18-PEG) or alternative.

In certain embodiments, the polyethylene glycol phospholipid is about between 4-8% of the lipid nanoparticle by molecular ratio or about between 5-7% of the lipid nanoparticle by molecular ratio.

In certain embodiments, the polyethylene glycol phospholipid has polyethylene glycol chains with an average molecule weight of about between 1700 and 2300 Da, or an average molecule weight of about between 1800 and 2200 Da, or an average molecule weight of about between 1900 and 2100 Da.

In certain embodiments, the ionizable lipid is about between 32-34% of the lipid nanoparticle by molecular ratio, wherein the phospholipid of about between 45-47% of the lipid nanoparticle by molecular ratio, wherein the sterol of about between 17-19% of the lipid nanoparticle by molecular ratio, and wherein the polyethylene glycol phospholipid is about between 2-4% of the lipid nanoparticle.

In certain embodiments, the lipid nanoparticles comprise an ionizable lipid which is (2S,2′S)-1,1′-((2-(((S)-2-hydroxydodecyl)(2-(4-(2-(((S)-2-hydroxydodecyl)((S)-2-hydroxydodecyl)amino)ethyl)piperazin-1-yl)ethyl)amino)ethyl)azanediyl)bis(dodecan-2-ol) (KB-S) or 2R,2′R)-1,1′-((2-(((R)-2-hydroxydodecyl)(2-(4-(2-(((R)-2-hydroxydodecyl)((R)-2-hydroxydodecyl)amino)ethyl)piperazin-1-yl)ethyl)amino)ethyl)azanediyl)bis(dodecan-2-ol) (KB-R) of between 32-34% of the lipid nanoparticle by molecular ratio; a phospholipid which is 1,2-dioleoyl-sn-glycero-3-phosphoethanolamine (DOPE) of between 45-47% of the lipid nanoparticle by molecular ratio; a sterol which is cholesterol of between 17-19% of the lipid nanoparticle by molecular ratio; and a polyethylene glycol phospholipid which is poly(ethylene glycol)-distearoyl phosphatidylethanolamine (C18-PEG) of between 2-4% of the lipid nanoparticle wherein the polyethylene glycol phospholipid has polyethylene glycol chains with an average molecule weight of about between 1900 and 2100 Da.

In certain embodiments, the diameter of the lipid nanoparticles is an average of about 140 to 160 nanometers.

In certain embodiments, the therapeutic peptide is an enzyme such as a purine nucleoside cleaving enzyme. In certain embodiments, the purine nucleoside cleaving enzyme is a non-mammalian purine nucleoside phosphorylase (PNP) or nucleoside hydrolase (NH).

In certain embodiments, lipid nanoparticles disclosed herein comprises a nucleic acid that encodes a therapeutic protein, an ionizable lipid, a phospholipid, a sterol, and a polyethylene glycol phospholipid.

In certain embodiments, the ionizable lipid is (2S,2′S)-1, l′-((2-(((S)-2-hydroxydodecyl)(2-(4-(2-(((S)-2-hydroxydodecyl)((S)-2-hydroxydodecyl)amino)ethyl)piperazin-1-yl)ethyl)amino)ethyl)azanediyl)bis(dodecan-2-ol) (KB-S) or 2R,2′R)-1,1′-((2-(((R)-2-hydroxydodecyl)(2-(4-(2-(((R)-2-hydroxydodecyl)((R)-2-hydroxydodecyl)amino)ethyl)piperazin-1-yl)ethyl)amino)ethyl)azanediyl)bis(dodecan-2-ol) (KB-R) or alternative.

In certain embodiments, the ionizable lipid is about between 25-40% of the lipid nanoparticle by molecular ratio, or about between 30-36% of the lipid nanoparticle by molecular ratio, or about between 32-34% of the lipid nanoparticle by molecular ratio.

In certain embodiments, the phospholipid is 1,2-dioleoyl-sn-glycero-3-phosphoethanolamine (DOPE) or alternative.

In certain embodiments, the phospholipid is about between 40-50% of the lipid nanoparticle by molecular ratio, or about between 44-48% of the lipid nanoparticle by molecular ratio, or about between 45-47% of the lipid nanoparticle by molecular ratio.

In certain embodiments, the sterol is cholesterol or alternative.

In certain embodiments, the sterol is about between 15-21% of the lipid nanoparticle by molecular ratio, or about between 16-20% of the lipid nanoparticle by molecular ratio, or about between 17-19% of the lipid nanoparticle by molecular ratio.

In certain embodiments, the polyethylene glycol phospholipid is poly(ethylene glycol)-distearoylphosphatidylethanolamine (C18-PEG) or alternative.

In certain embodiments, the polyethylene glycol phospholipid is about between 1-5% of the lipid nanoparticle by molecular ratio or about between 2-4% of the lipid nanoparticle.

In certain embodiments, the polyethylene glycol phospholipid has polyethylene glycol chains with an average molecule weight of about between 1700 and 2300 Da, or an average molecule weight of about between 1800 and 2200 Da, or an average molecule weight of about between 1900 and 2100 Da.

In certain embodiments, lipid nanoparticles disclosed herein can be formulated to target a specific cell type or tissue.

In certain embodiments, the disclosed lipid nanoparticles include an ionizable lipid or alternative. Ionizable lipids have a positive or partial positive charge at physiological pH. Exemplary alternative ionizable lipids include but are not limited to 3,6-bis({4-[bis(2-hydroxydodecyl)amino]butyl})piperazine-2,5-dione (cKK-E12), 1-Linoleoyl-2-linoleyloxy-3-dmiethylaminopropane (DLin-2-DMAP), 1,2-Dilinoleylcarbanioyloxy-3-dimethylaniinopropane (DLin-C-DAP), 1,2-Dilmoleoyl-3-dimethylammopropane (DLm-DAP), 1,2-Dilinoleyloxy-N,N-dimethylaminopropane (DLin-DMA), 2,2-Dilinoleyl-4-dimethy laminomethyl-[1,3]-dioxolane (DLin-K-DMA), 2,2-dilmoleyl-4-(2-dimethylaiiimoethyl)-[1,3]-dioxolane (DLin-KC2-DMA), (6Z,9Z,28Z,31Z)-heptatriaconta-6,9,28,31-tetraen-19-yl 4-(dimethylamino) butanoate (DLin-MC3-DMA), 1,2-dioieoyl-3-dimethylammonium propane (DODAP), N,N-dimethyl-(2,3-dioleyloxy) propylamine (DODMA), dioctadecylamidoglycyocarboxysperrnine (DOGS), Spermine cholesterylcarbamate (GL-67), bis-guanidinium-spermidine-cholesterol (BGTC), N-t-butyl-N′-tetradecylamino-propionamidine (diC14-amidine), Dimethyldioctadecylammoniurn bromide (DDAB), N-(1,2-dimyristyloxyprop-3-yl)-N,N-dimethyl-N-hydroxyethyl ammonium bromide (DMRIE), N, N-dioleyl-N,N-dimethylammonium chloride (DODAC), Dioleyloxypropyl-3-dimethyl hydroxyethyl ammonium bromide (DORIE), N-(1-(2,3-dioleyloxy3) propyl)-N-2-(spenninecarboxamido)ethyl)-N,N-dimethylamrnonium trifluoracetate (DOSPA), 2-dioleoy trimethyl ammonium propane chloride (DOTAP), N-(1-(2,3-dioleyloxy) propyl)-N,N,N-trimethylammonium chloride (DOTMA), Aminopropyl-dimethyl-bis(dodecyloxy)-propanaminiumbromide (GAP-DLRIE), 1,2-dioleoyl-sn-3-phosphoethanolamine (“DOPE”), or combinations thereof.

In certain embodiments, the disclosed lipid nanoparticles include a polyethylene glycol phospholipid or alternative. Such polyethylene glycol phospholipid may be alternately referred to as PEGylated lipids. Inclusion of a PEGylating lipid can be used to enhance lipid nanoparticle colloidal stability in vitro and circulation time in vivo. In some embodiments, the PEGylation is reversible in that the PEG moiety is gradually released in blood circulation. Exemplary PEG-lipids include but are not limited to PEG conjugated to saturated or unsaturated alkyl chains having a length of C3-C22. PEG-modified phosphatidylethanolamines, PEG-modified phosphatidic acids, PEG-modified ceramides (PEG-CER), PEG-modified dialkylamines, PEG-modified diacylglycerols (PEG-DAG), PEG-modified dialkylglycerols, and mixtures thereof. For example, a PEG lipid may be PEG-c-DOMG, PEG-DMG, PEG-DLPE, PEG-DMPE, PEG-DPPC or a PEG-DSPE lipid. In certain embodiments, the molecular weight of the PEG lipid can be about 1 KDa, 2 KDa, or 3 KDa.

In certain embodiments, the disclosed lipid nanoparticles include an alternative phospholipid moiety that may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Nonnatural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer to provide a nanoparticle composition with a targeting or imaging moiety (e.g., a dye).

In certain embodiments, the disclosed lipid nanoparticles include alternative phospholipids such as 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoy 1-sn-glycero-3-phosphocholine (OChemsPC), 1-hexadecyl-sn-glycero-3-phosphocholine (CI 6 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidylethanolamine (DSPE), dipalmitoyl phosphatidyl ethanolamine (DPPE), dimyristoylphosphoethanolamine (DMPE), 1-stearoyl-2-oleoyl-phosphatidy ethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, or lysophosphatidylethanolamine (LPE).

### Cancer Therapies

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation of reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

The term “effective amount” refers to that amount of a compound or pharmaceutical composition described herein that is sufficient to induce the intended application including, but not limited to, disease treatment, as illustrated below. In relation to a combination therapy, an “effective amount” indicates the combination of agent results in synergistic or additive effect when compared to the agents individually. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compound(s) chosen, the dosing regimen to be followed, whether it is administered in combination with other agents, timing of administration, the tissue to which it is administered, and the physical delivery system in which it is carried.

The cancer to be treated in the context of the present disclosure may be any type of cancer or tumor (or related condition) such as head and neck squamous cell carcinoma (HNSCC), lung cancer, non-small cell lung cancer (NSCLC) and subtypes such as adenocarcinoma, squamous cell carcinoma, and large cell carcinoma, and small cell lung cancer. Contemplated are malignancies located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax and genito-urinary apparatus and, more particularly, adrenocortical carcinoma, AIDS-related lymphoma, AIDS-related malignant tumors, anal cancer, astrocytoma, cancer of the biliary tract, cancer of the bladder, bone cancer, brain stem glioma, brain tumors, breast cancer, primary central nervous system cerebellar astrocytoma, brain astrocytoma, cancer of the cervix, chronic lymphocytic leukemia, chronic myeloid leukemia, cancer of the colon, cutaneous T-cell lymphoma, endocrine pancreatic islet cell carcinoma, endometrial cancer, ependymoma, epithelial cancer, cancer of the esophagus, Ewing's sarcoma and related tumors, cancer of the exocrine pancreas, extracranial germ cell tumor, extragonadal germ cell tumor, extrahepatic biliary tract cancer, cancer of the eye, Gaucher's disease, cancer of the gallbladder, gastric cancer, gastrointestinal carcinoid tumor, gastrointestinal tumors, germ cell tumors, gestational trophoblastic tumor, head and neck cancer, hepatocellular cancer, hypergammaglobulinemia, hypopharyngeal cancer, Hodgkin's disease, intestinal cancers, intraocular melanoma, islet cell carcinoma, islet cell pancreatic cancer, Kaposi's sarcoma, cancer of the larynx, cancer of the lip and mouth, macroglobulinemia, malignant mesothelioma, malignant thymoma, medulloblastoma, melanoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-Hodgkin's lymphoma, non-melanoma skin cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, pancreatic cancer, paraproteinemias, purpura, cancer of the penis, hypophysis tumor, primary central nervous system lymphoma, primary liver cancer, prostate cancer, rectal cancer, renal cell cancer, cancer of the renal, pelvis, and ureter, retinoblastoma, rhabdomyosarcoma, cancer of the salivary glands, sarcoidosis, sarcomas, small intestine cancer, soft tissue sarcoma, squamous neck cancer, stomach cancer, pineal and supratentorial primitive neuroectodermal tumors, testicular cancer, thymoma, trophoblastic tumors, cancer of the urethra, cancer of the uterus, uterine sarcoma, vaginal cancer, optic pathway and hypothalamic glioma, cancer of the vulva, Waldenstrom's macroglobulinemia, Wilm's tumor and any other hyperproliferative disease, as well as neoplasia, located in the system of a previously mentioned organ.

In certain embodiments, compounds disclosed herein may be administered in combination with an additional anti-cancer agent. A “chemotherapy agent,” “chemotherapeutic,” “anti-cancer agent” or the like, refer to molecules that are recognized to aid in the treatment of a cancer. Contemplated examples include the following molecules or derivatives such as abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); adriamycin, bleomycin, vinblastine, dacarbazine (ABVD); cyclophosphamide, doxorubicin, vincristine, prednisolone (CHOP); rituximab, cyclophosphamide, doxorubicin, vincristine, prednisolone (RCHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC). In certain embodiments, the chemotherapy agent is an antibody, anti-PD-1, anti-PD-L1, anti-CTLA4 antibody or combinations thereof, such as an anti-CTLA4 (e.g., ipilimumab, tremelimumab), an anti-PD-L1 (e.g., atezolizumab, avelumab, durvalumab) or an anti-PD1 antibody (e.g., nivolumab, pembrolizumab, cemiplimab, dostarlimab, spartalizumab, camrelizumab, tislelizumab, toripalimab, sintilimab).

In certain embodiments, this disclosure relates to methods of treating cancer comprising administering an effective amount of a lipid nanoparticle as disclosed herein to a subject in need thereof, wherein the lipid nanoparticle comprises a vector or nucleic acid encoding a peptide based anticancer agent. In certain embodiments the peptide based anticancer agent is a single chain antibody.

In certain embodiments, cancer treatment methods disclosed herein may be done in combination with administering a chemotherapy agent or combination of chemotherapy agents, radiation therapy or surgical intervention.

In certain embodiments, the peptide based anticancer agent is a single chain antibody. In certain embodiments, the single chain antibody specifically binds PD-1, PD1-L, or CTLA-4.

In certain embodiments, this disclosure relates to intravenous or intratumoral injection of nanoparticles disclosed herein comprising a single chain antibody. In certain embodiment, this disclosure relates to the use of the single chain antibody to increase anti-cancer activity of immune-type therapeutic agents. In certain embodiments, immune-type therapies include checkpoint blockade inhibitors including CTLA-4 blockers, PD1 antibodies, PD1 ligand antibodies, or T- or B-cell therapies.

In certain embodiment, this disclosure relates to the use of lipid nanoparticles comprising a nucleic acid, DNA, RNA, or vector encoding a therapeutic protein as disclosed herein followed by tumor regression (or other gene therapy-based molecular chemotherapies) and/or as a means to provide synergy with other anticancer agents or therapies.

In certain embodiments, this disclosure relates to methods of treating cancer or initiating, enhancing, or prolonging an anti-tumor response in a subject in need thereof comprising administering to the subject an effective number of lipid nanoparticles comprising a nucleic acid encoding a therapeutic protein, antibody, antibody fragment, a single chain antibody, or peptide based anticancer agent as disclosed herein.

In certain embodiments, the peptide based anticancer agent is a purine nucleoside cleaving enzyme (e.g., a nucleoside phosphorylase such as E. coli PNP), and administering a prodrug cleaved by said purine nucleoside cleaving enzyme to the subject.

In certain embodiments, the purine nucleoside cleaving enzyme is a non-mammalian purine nucleoside phosphorylase (PNP) or nucleoside hydrolase (NH).

In certain embodiments, the prodrug is 9-(β-D-arabinofuranosyl)-2-fluoroadenine (F-araA), 2-F-2′-deoxyadenosine (F-dAdo), fludarabine phosphate (F-araAMP, 2-fluoro-9-(5-O-phosphono-β-D-arabinofuranosyl)-9H-purin-6-amine), derivative, or salt thereof.

In certain embodiments, cancer treatment methods performed herein may be done in combination with administering another anticancer agent to the subject.

In certain embodiments, the purine nucleoside cleaving enzyme is a non-mammalian purine nucleoside phosphorylase (PNP) or nucleoside hydrolase (NH). In certain embodiments, the nucleic acid is DNA, RNA, or vector encoding the therapeutic protein.

In certain embodiment, the prodrug is 9-(β-D-arabinofuranosyl)-2-fluoroadenine (F-araA), 2-F-2′-deoxyadenosine (F-dAdo), fludarabine phosphate (F-araAMP, 2-fluoro-9-(5-O-phosphono-β-D-arabinofuranosyl)-9H-purin-6-amine), or a derivative, or salt thereof.

In certain embodiments, this disclosure relates to methods of treating cancer or initiating, enhancing, or prolonging an anti-tumor response in a subject in need thereof comprising administering to the subject an effective amount of lipid nanoparticles disclosed herein comprising a nucleic acid encoding a purine nucleoside cleaving enzyme, a purine nucleoside phosphorylase or nucleoside hydrolase in the absence of a prodrug cleaved by said purine nucleoside cleaving enzyme.

In certain embodiments, this disclosure relates to administering to the subject lipid nanoparticles comprising a nucleic acid encoding a therapeutic protein, a purine nucleoside phosphorylase or nucleoside hydrolase, given by intravenous or direct injection of the lipid nanoparticles into replicating or non-replicating targeted cells and optional exposure of the targeted cells to X-ray radiation.

In certain embodiments, the said replicating or non-replicating targeted cells are cancerous or define a tumor. In certain embodiment, the nucleic acid is DNA, RNA, messenger RNA, vector, or viral vector. In certain embodiments, said viral vector is an adenoviral vector or lentiviral vector. In certain embodiment, purine nucleoside phosphorylase is derived from E. coli or T. vaginalis or other bacterial strains. In certain embodiment, purine nucleoside phosphorylase is a mutant of E. coli PNP.

In certain embodiments, this disclosure relates to intravenous or intratumoral injection of nanoparticles disclosed herein comprising a nucleic acid or vector for expression of E. coli PNP followed by systemic prodrug treatment. In certain embodiment, this disclosure relates to the use of intratumoral expression of E. coli PNP or other non-mammalian proteins to increase anti-cancer activity of immune-type therapeutic agents. In certain embodiments, immune-type therapies include checkpoint blockade inhibitors including CTLA-4 blockers, PD1 antibodies, PD1 ligand antibodies, or T- or B-cell therapies.

In certain embodiment, this disclosure relates to the use of lipid nanoparticles comprising a nucleic acid, DNA, RNA, or vector encoding a therapeutic protein or PNP protein as disclosed herein in combination with intratumoral PNP expression followed by nucleoside-mediated tumor regression (or other gene therapy-based molecular chemotherapies) as a means to provide synergy with other anticancer agents or therapies.

### Pharmaceutical Compositions

In certain embodiments, this disclosure relates to pharmaceutical compositions containing lipid nanoparticles provided herein and optionally a pharmaceutically excipient or carrier. In certain embodiments, the lipid nanoparticle compositions may be formulated in whole or in part as pharmaceutical compositions. In certain embodiments, a pharmaceutical composition may include one or more nanoparticle compositions including one or more different therapeutic agents/anticancer agents. Pharmaceutical compositions may further include one or more pharmaceutically acceptable excipients or accessory ingredients such as those described herein.

In certain embodiments, the pharmaceutical compositions containing lipid nanoparticles disclosed herein can be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration and can be formulated in dosage forms appropriate for each route of administration.

In certain embodiments, pharmaceutical compositions containing lipid nanoparticles disclosed herein, may be administered locally, for example by injection directly into a tumor or tissue to be treated. Typically, the injection causes an increased localized concentration of the desired peptide expression which is greater than that which can be achieved by systemic administration. In certain embodiments, pharmaceutical composition containing lipid nanoparticles can be combined with a matrix, e.g., collagen mesh or sponge, to assist in creating an increased localized concentration of the polypeptide compositions by reducing the passive diffusion of the polypeptides out of the site to be treated.

In certain embodiments, the pharmaceutical composition containing lipid nanoparticles disclosed herein is administered in solvent, e.g., an aqueous solution, by parenteral injection. The formulation may also be in the form of a suspension or emulsion. In certain embodiments, the pharmaceutical composition containing lipid nanoparticles disclosed herein optionally includes pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., ascorbic acid, sodium metabisulfite), and preservatives (e.g., benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through filters that a sterile effluent even when loaded with bacteria, by incorporating sterilizing agents into the compositions, by irradiating the compositions, or by heating the compositions.

### High-Throughput DNA Barcoding to Identify Optimal Candidates for the Delivery of LNPs to Tumor Cells In Vivo

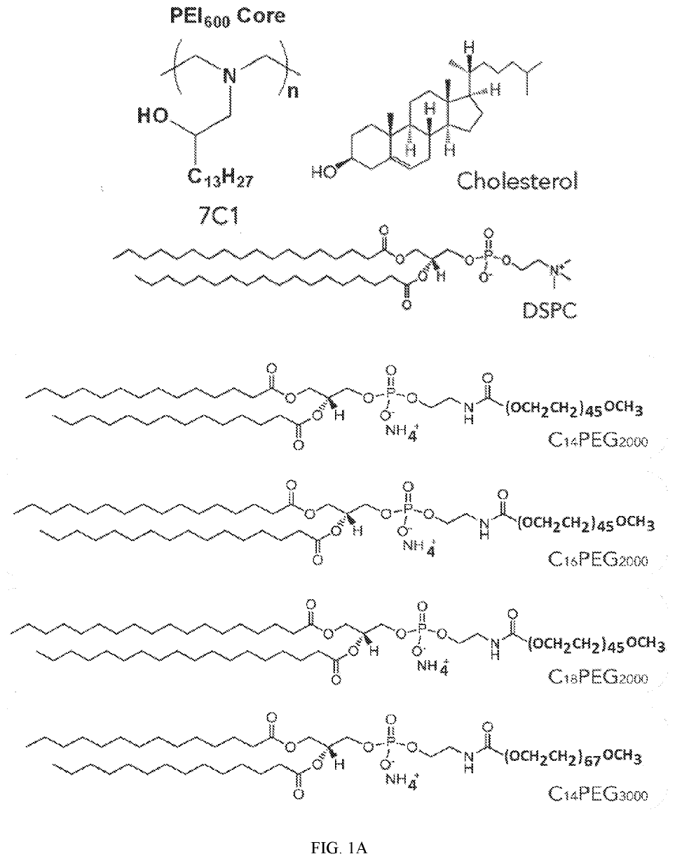

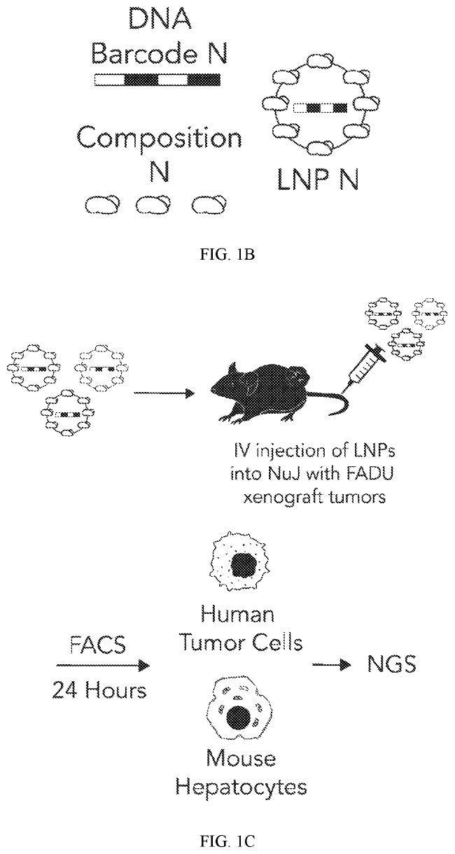

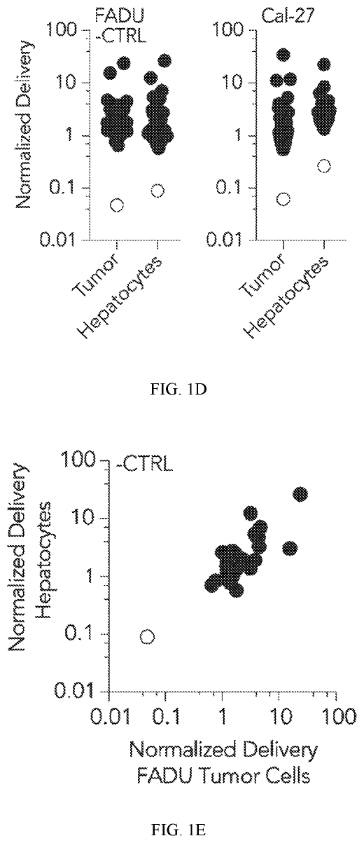

The improved delivery of LNPs to human tumor cell-types in vivo was explored using a high throughput functional DNA barcoding screen. Different LNPs were formulated using the ionizable lipid 7C1, cholesterol, DSPC helper lipid, and PEG-lipids with varying molar amounts, alkyl lengths, and molecular weights (FIG. 1A). Each distinct LNP carried a unique DNA barcode (FIG. 1B). LNP 1, comprised of material 1, was formulated with barcode 1; LNP N, comprised of material N, was formulated with barcode N. Quality control was run on each LNP using dynamic light scattering (DLS). To pass quality control steps, LNPs were small (between 20-200 nm) and monodisperse. LNPs that followed these criteria were pooled together.

The pool was injected intravenously (IV) into two groups of immunocompromised Nu/J mice at a dose of 0.5 mg/kg of DNA barcode (FIG. 1C). These two groups were previously injected with two different human cancer cell lines (FADU and Cal-27 human tumor cell lines) to establish hindleg xenograft tumors. Each mouse had one tumor per leg, resulting in two tumors inoculated with LNPs per mouse. Twenty-four hours later, human tumor cells and liver hepatocytes were isolated via fluorescently activated cell sorting (FACS). DNA barcodes were isolated and quantified via next-generation sequencing (NGS). Normalized DNA delivery (%) from tumor cells and hepatocytes for each of tumor types were plotted (FIG. 1D). As a control (open circle), an unencapsulated barcode was found to be poorly delivered compared to barcodes within an LNP (FIG. 1D). To identify LNP traits that are preferentially delivered to human tumors, hepatocyte delivery against human tumor cell delivery was plotted across both tumor types (FIGS. 1E and 1F). Three LNPs, deemed tumor-specific LNPs, that preferentially deliver to human tumor cells were identified.

Based on the LNPs identified in the first screen, the compositional makeup of three tumor-specific LNPs were identified for further evaluation. All three LNPs contained C18PEG2000 (FIG. 2A). C18PEG2000 was predominantly enriched in the top 5% LNPs. Similarly, 7C1 at 50% mole ratio was predominantly enriched. Based on these findings, a second screen was designed around C18PEG2000 and 7C1 at a 50% molar ratio. In this second screen, different 7C1-based LNPs were formulated with unique DNA barcodes, with the same methodology as the first screen. Two groups of immunocompromised mice were treated with either Cal-27 or FADU human tumor cell lines (FADU and Cal-27) to create hindleg xenograft tumors (one tumor per hindleg). All mice were injected with the stable LNP pool at a dose of 0.5 mg/kg of DNA barcode. Human tumor cells and hepatocytes where isolated with FACS and barcodes were sequenced. DNA barcodes across tumor cells and hepatocytes in both cell lines were quantified (FIG. 2B). From this experiment, the normalized delivery levels in hepatocytes were plotted against normalized delivery in human tumor cells (FIG. 2C, 2D). A lipid nanoparticle capable of delivering nucleic acid selectively to tumor cells with optimal pharmacological properties was identified. Designated as Tumor Lipid Nanoparticle 1 (TL1), it provided the highest delivery to tumor cells but also minimized delivery to hepatocytes. It is desirable to minimized hepatic delivery. TL1 contains: cholesterol (28.5%), 7C1 (50%), DSPC (15%), and C18PEG2000 (6.5%).

Tumor LNP can Deliver mRNA to Tumor Cell Types In Vitro

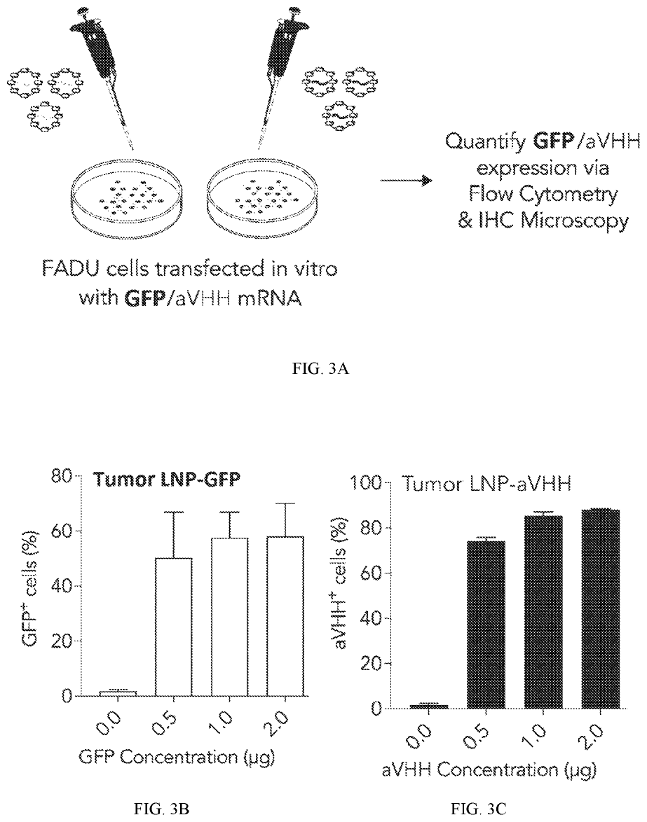

Experiments were performed to determine whether TL1 could deliver a relevant nucleic acid payload (i.e., mRNA) to human head and neck cancer cells. GFP- and anchored VHH (aVHH)-encoding mRNA was encapsulated in TL1. FADU cancer cells were transfected with the nanoparticles and cultured in vitro (FIG. 3A). Using flow cytometry, it was determined that around 60% and 80% of the FADUs expressed GFP and aVHH, respectively (FIGS. 3B and 3C). GPI-anchored camelid-derived single chain antibody (aVHH) transfection was verified via immunohistochemical staining. These experiments indicate that one can use TL1 to transfect human head and neck cancer cells independently of the mRNA payload.

Tumor LNP can Deliver mRNA to Tumor Cell Types In Vivo

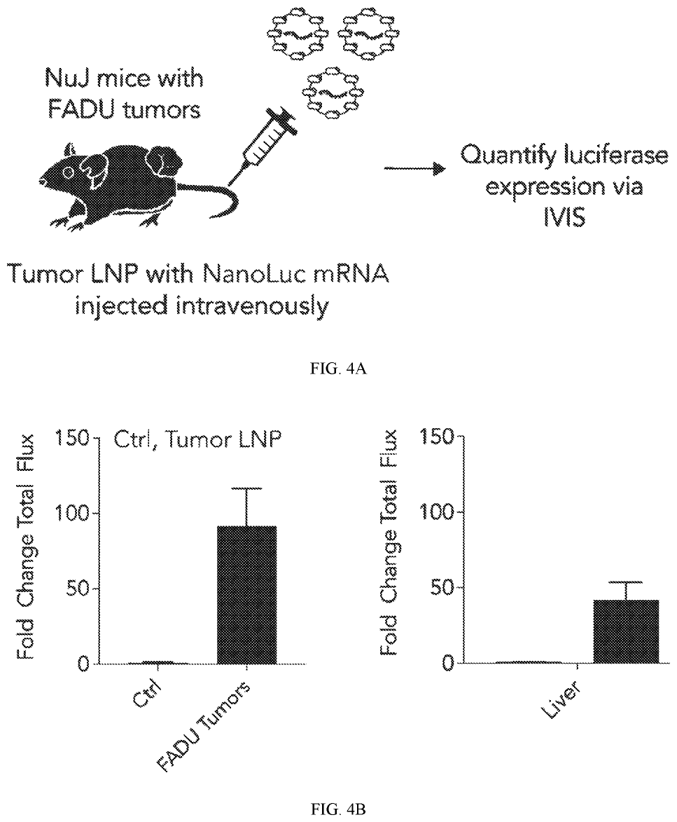

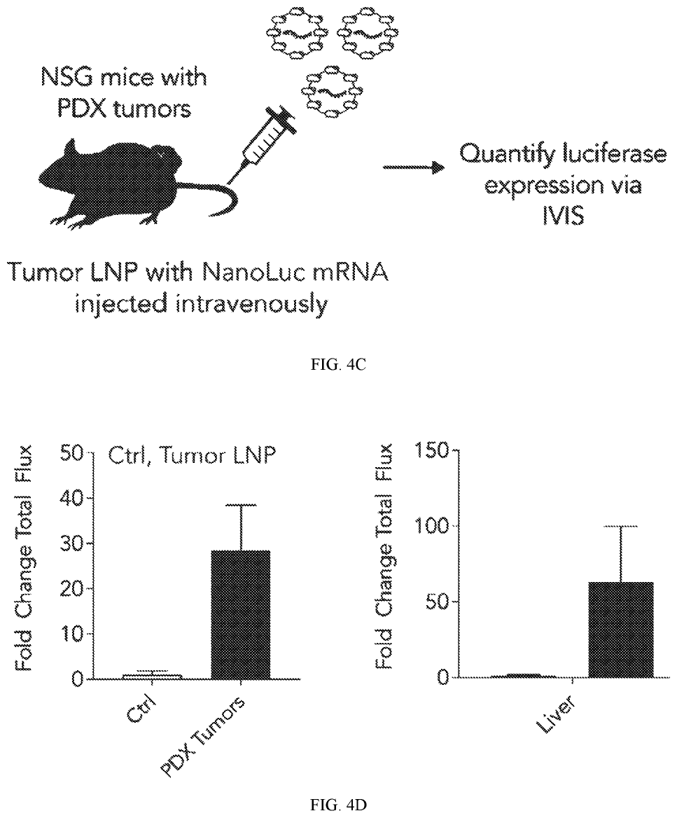

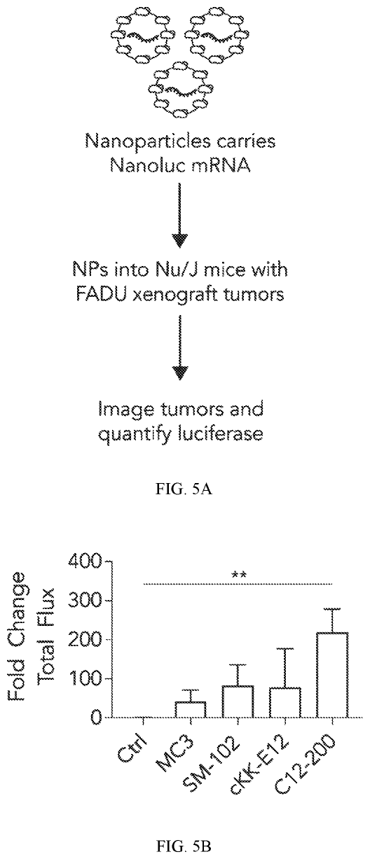

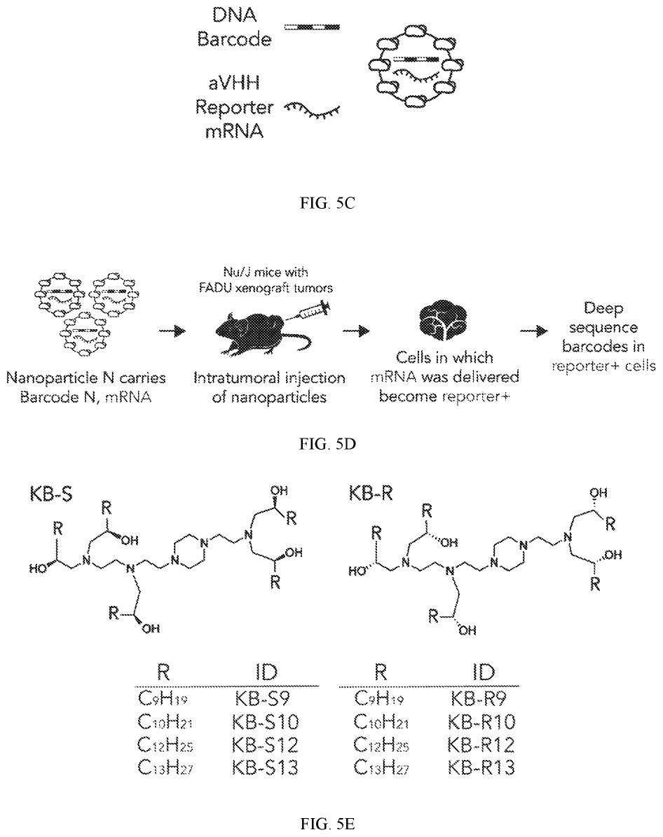

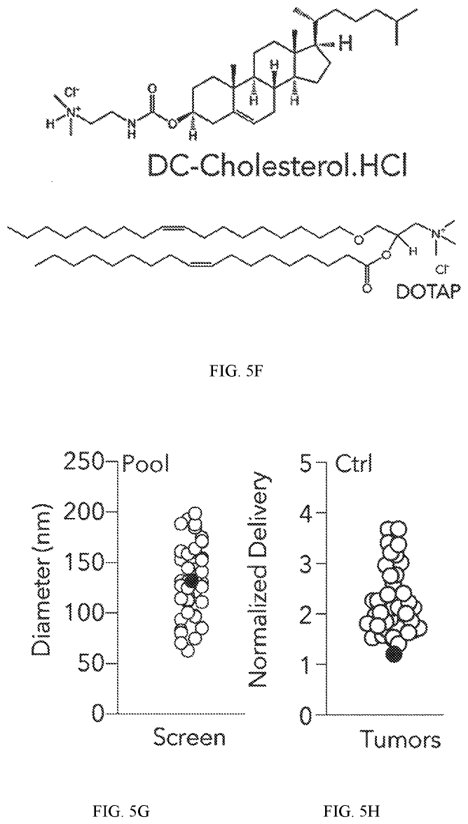

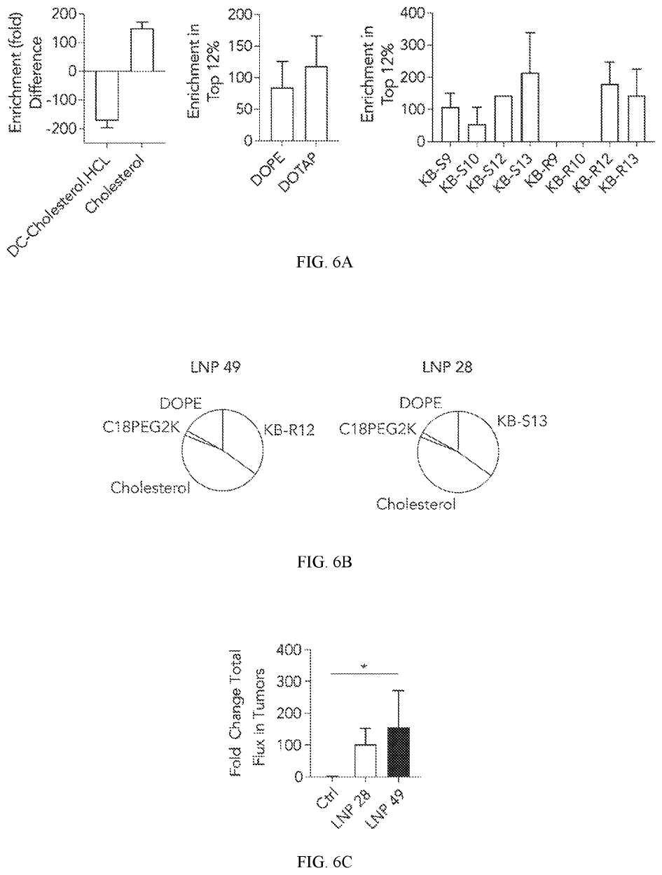

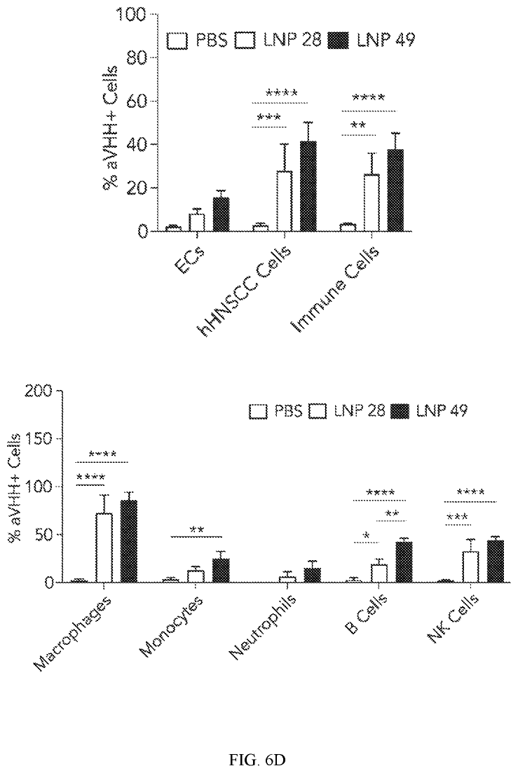

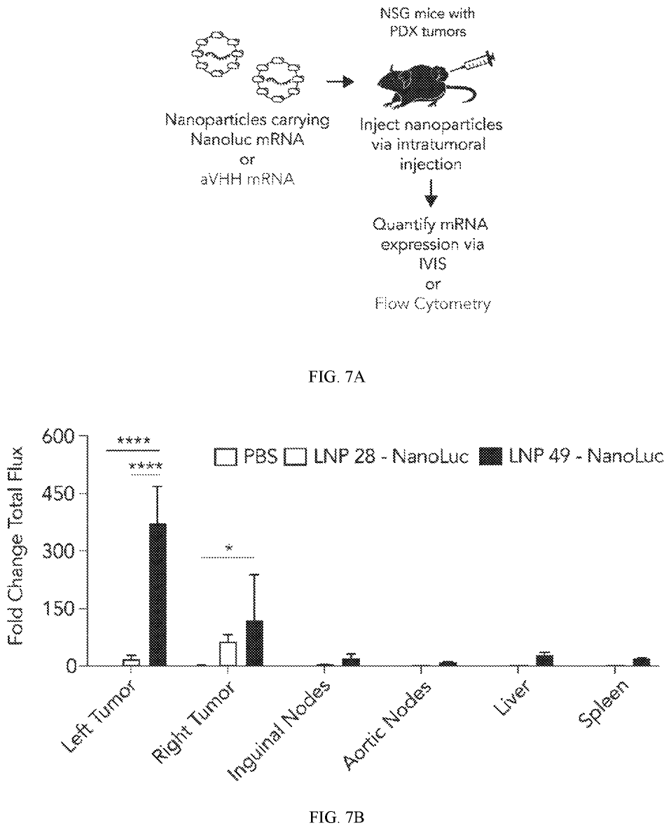

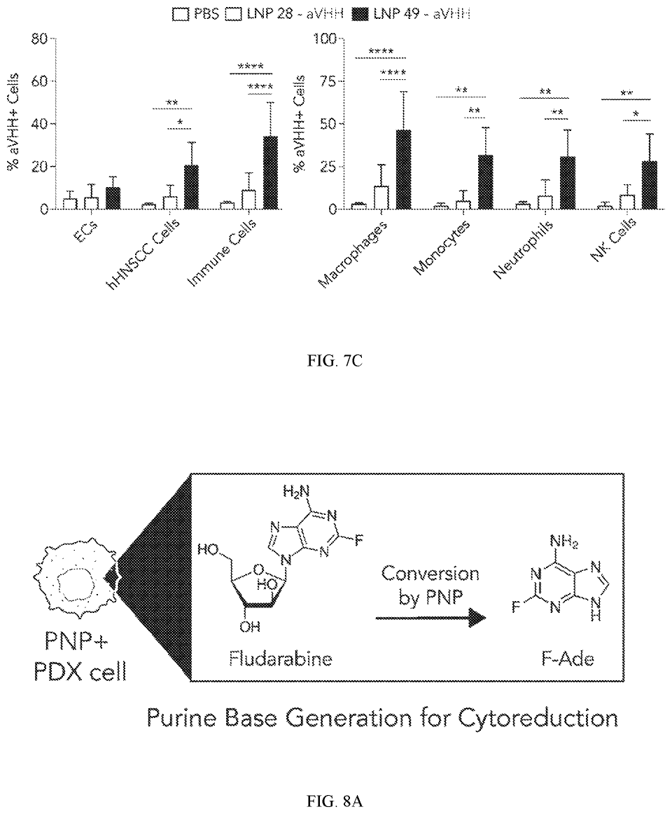

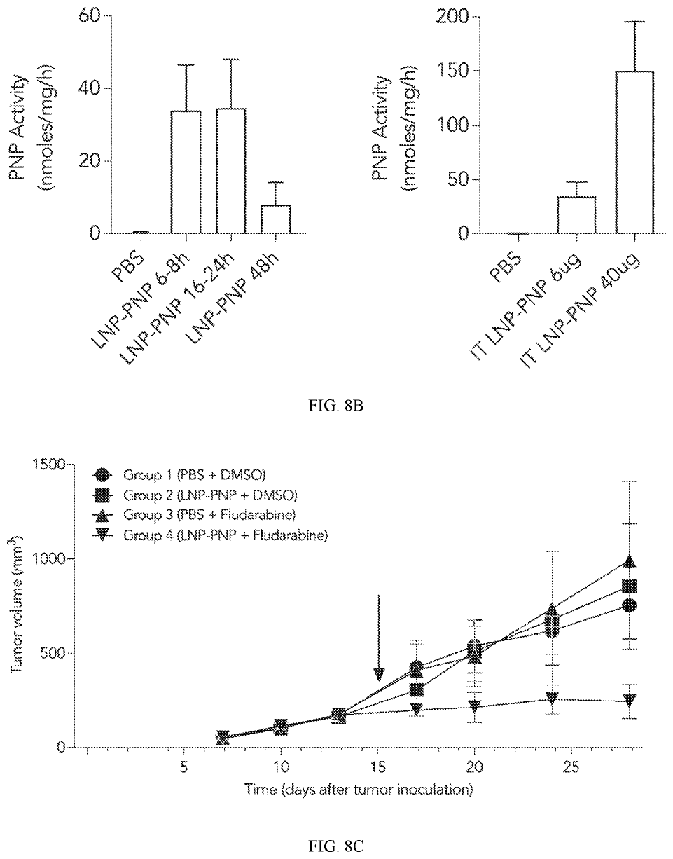

To test mRNA delivery using TL1 in vivo, mice were inoculated with hindleg flank tumors. Two FADU xenograft tumors were inoculated in immunocompromised Nu/J mice, one per leg (FIG. 4A). TL1 was formulated with an mRNA that encodes an anchored nanoluciferase (anNanoLuc). Mice with FADU flank tumors were intravenously administered with TL1 at a dose of 2 mg/kg anNanoLuc mRNA. As a control, mice were injected with 1×PBS. Forty-eight hours later, the tumors were harvested and evaluated to quantify luciferase expression by measuring whole-organ bioluminescence. Statistically higher bioluminescence was obtained in both the right- and left-side of FADU tumor xenografts, compared to control tumors (FIG. 4B). Higher bioluminescence was observed in the tumors compared to that in the liver, thus demonstrating that TL1 selectively targeted human cancer cells over liver hepatocytes. Immunocompromised mice were inoculated with bilateral patient-derived xenograft (PDX) tumors, one per leg, and injected with an NanoLuc-carrying TL1 intravenously (FIG. 4C). Both PDX tumors were bioluminescent, and the liver displayed minimal bioluminescence (FIG. 4D) demonstrating that TL1 can successfully transfect primary human cancer cells in vivo.

What is claimed is:

1. A pharmaceutical composition comprising lipid nanoparticles comprising,
a nucleic acid that encodes a therapeutic protein,
an ionizable lipid,
a phospholipid,
a sterol, and
a polyethylene glycol phospholipid.

2. The pharmaceutical composition of claim 1, wherein the therapeutic protein is a single chain antibody.

3. The pharmaceutical composition of claim 1, wherein the ionizable lipid is 7C1.

4. The pharmaceutical composition of claim 3, wherein the ionizable lipid is about between 40-60% of the lipid nanoparticle by molecular ratio, or about between 45-55% of the lipid nanoparticle by molecular ratio, or about between 48-52% of the lipid nanoparticle by molecular ratio.

5. The pharmaceutical composition of claim 1, wherein the phospholipid is distearoylphosphatidylcholine (DSPC).

6. The pharmaceutical composition of claim 5, wherein the phospholipid is about between 10-20% of the lipid nanoparticle by molecular ratio, or about between 12-17% of the lipid nanoparticle by molecular ratio, or about between 14-16% of the lipid nanoparticle by molecular ratio.

7. (canceled)

8. The pharmaceutical composition of claim 1, wherein the sterol is about between 25-32% of the lipid nanoparticle by molecular ratio, or about between 26-31% of the lipid nanoparticle by molecular ratio, or about between 27-30% of the lipid nanoparticle by molecular ratio.

9. The pharmaceutical composition of claim 1, wherein the polyethylene glycol phospholipid is poly(ethylene glycol)-distearoylphosphatidylethanolamine (C18-PEG).

10. The pharmaceutical composition of claim 1, wherein the polyethylene glycol phospholipid is about between 4-8% of the lipid nanoparticle by molecular ratio or about between 5-7% of the lipid nanoparticle.

11. (canceled)

12. The pharmaceutical composition of claim 1, wherein the ionizable lipid is 7C1 of about between 48-52% of the lipid nanoparticle by molecular ratio, wherein the phospholipid is distearoylphosphatidylcholine (DSPC) of about between 14-16% of the lipid nanoparticle by molecular ratio, wherein the sterol is cholesterol of about between 27-30% of the lipid nanoparticle by molecular ratio, and wherein the polyethylene glycol phospholipid is poly(ethylene glycol)-distearoylphosphatidylethanolamine of about between 5-7% of the lipid nanoparticle and wherein the polyethylene glycol chains have an average molecule weight average molecular of about between 1900 and 2100 Da.

13. The pharmaceutical composition of claim 12 wherein the diameter of the lipid nanoparticles is an average of about 140 to 160 nanometers.

14. The pharmaceutical composition of claim 1, wherein the therapeutic protein is a purine nucleoside cleaving enzyme.

15-24. (canceled)

25. The pharmaceutical composition of claim 1, wherein the ionizable lipid is (2S,2′S)-1,1′-((2-(((S)-2-hydroxydodecyl)(2-(4-(2-(((S)-2-hydroxydodecyl)((S)-2-hydroxydodecyl)amino)ethyl)piperazin-1-yl)ethyl)amino)ethyl)azanediyl)bis(dodecan-2-ol) (KB-S) or 2R,2′R)-1,1′-((2-(((R)-2-hydroxydodecyl)(2-(4-(2-(((R)-2-hydroxydodecyl)((R)-2-hydroxydodecyl)amino)ethyl)piperazin-1-yl)ethyl)amino)ethyl)azanediyl)bis(dodecan-2-ol) (KB-R) of between 32-34% of the lipid nanoparticle by molecular ratio; the phospholipid is 1,2-dioleoyl-sn-glycero-3-phosphoethanolamine (DOPE) of between 45-47% of the lipid nanoparticle by molecular ratio; wherein the sterol is cholesterol of between 17-19% of the lipid nanoparticle by molecular ratio; and the polyethylene glycol phospholipid is poly(ethylene glycol)-distearoyl phosphatidylethanolamine (C18-PEG) of between 2-4% of the lipid nanoparticle; and wherein the polyethylene glycol phospholipid has polyethylene glycol chains with an average molecule weight of about between 1900 and 2100 Da.

26. A method of treating cancer comprising administering an effective amount of a pharmaceutical composition of claim 1 comprising lipid nanoparticles encapsulating a vector or nucleic acid encoding a peptide based anticancer agent.

27. The method of claim 26, wherein the peptide based anticancer agent is a single chain antibody.

28. The method of claim 27, wherein the single chain antibody specifically binds PD-1, PD1-L, or CTLA-4.

29. The method of claim 26, wherein the peptide based anticancer agent is a purine nucleoside cleaving enzyme and administering a prodrug cleaved by said purine nucleoside cleaving enzyme to the subject.

30. The method of claim 29, wherein the purine nucleoside cleaving enzyme is a non-mammalian purine nucleoside phosphorylase (PNP) or nucleoside hydrolase (NH).

31. The method of claim 30, wherein the prodrug is 9-(β-D-arabinofuranosyl)-2-fluoroadenine (F-araA), 2-F-2′-deoxyadenosine (F-dAdo), fludarabine phosphate (F-araAMP, 2-fluoro-9-(5-O-phosphono-β-D-arabinofuranosyl)-9H-purin-6-amine), derivative, or salt thereof.

32. The method of claim 26 in combination with administering an anticancer agent to the subject.
